(19) United States
(12) Patent Application Publication
(10) Pub. No.: US 2017/0053078 A1
(43) Pub. Date: Feb. 23, 2017

(54) QUANTIFYING AND REPORTING USER READINESS

(71) Applicant: COVIDIEN LP, Mansfield, MA (US)

(72) Inventors: AARON JOHN LANZEL, Annapolis, MD (US); BENJAMIN DAVID MORRIS, Annapolis, MA (US); BRIAN KEITH RUSSELL, Annapolis, MD (US)

(21) Appl. No.: 15/239,405

(22) Filed: Aug. 17, 2016

Related U.S. Application Data
(60) Provisional application No. 62/206,639, filed on Aug. 18, 2015.

(51) Int. Cl. G06F 19/00; A61B 5/024; G06N 7/00

(52) U.S. Cl. CPC G06F 19/345; G06N 7/005; A61B 5/024

(57) ABSTRACT

Methods, apparatuses and systems are described for determining a readiness of a user. The methods may include receiving physiological data corresponding to one or more physiological parameters of the user. The methods may also include receiving user input data corresponding to one or more subjective user state parameters of the user. The methods may also include assigning a respective component score to each of the received physiological data and user input data. The methods may further include assigning a respective weight to at least one of the one or more physiological parameters or one or more subjective user state parameters of the user. The methods may also include deriving a readiness score for the user based at least in part on the component scores of the corresponding weights. The derived readiness score may be communicated to the user via a display device.

### CROSS REFERENCE

The present Application for Patent claims priority to U.S. Provisional Patent Application No. 62/206,639 by Lanzel et al., entitled “Quantifying and Reporting User Readiness,” filed Aug. 18, 2015, assigned to the assignee hereof.

### BACKGROUND

The present disclosure relates generally to physiological monitoring systems, and more particularly to deriving a readiness score providing a numerical prediction representative of a user's overall health and/or preparedness for physical activity.

Use of mobile personal monitoring devices in sports and physical activity applications is well known, but many of these activity monitors may be limited in their functionality to providing direct measurements of various physiological parameters, such as heart rate or number of steps taken, either as the activity is occurring or after the fact, rather than providing any predictive data. Further, the data monitored may be limited to one or more separate parameters, and may therefore fail to provide context for the overall health or wellness of the monitored user.

Additionally, while monitored physiological parameters are a key element of determining the overall wellness of a monitored user, user health reports limited to detected physiological data alone may provide an incomplete picture of the user's health status. Additional, subjective components may be required to provide a more comprehensive view of the user's physical status.

### SUMMARY

Predictive health and wellness reports may be particularly useful to coaches and healthcare providers, in order to project a monitored user's health status or readiness for an upcoming day or athletic event. It may be particularly beneficial to provide a numerical representation of the user's readiness, such that the user's health status may be quickly and easily obtained and compared with that of other users, or compared with the monitored user's previously observed readiness levels. Furthermore, it may be beneficial to provide a more holistic representation of the user's health status by incorporating subjective health parameters, such as quality of sleep, level of stress, and the like, when deriving a numerical representation of the user's readiness. One method of accomplishing this may include receiving physiological data corresponding to one or more physiological parameters of the user, and receiving user input data corresponding to one or more subjective user state parameters of the user. The method may further include assigning a respective component score to each of the received physiological data and user input data. The method may also include assigning a respective weight to at least one of the one or more physiological parameters or one or more subject user state parameters of the user. A readiness score may then be derived for the user based at least in part on the component scores and the corresponding weights, and the derived readiness score may be communicated to the user via a display device.

The respective component scores assigned to each of the received physiological data and user input data may be predetermined based at least in part on individual user physiological conditions. In this way, monitored physiological and subjective health data may be tailored to individual user health, and may be more accurately representative of the monitored user's health status.

In some embodiments, deriving the readiness score may further include calculating a weighted average of the respective component scores for each of the received physiological data and user input data, where the respective component scores are weighted by respective weights.

In some embodiments, the method for determining the readiness of the user may further include receiving recorded data corresponding to the one or more subjective user state parameters of the user, and replacing the received user input data with the received recorded data.

In some embodiments, the respective component score assigned to each of the received physiological data and user input data may be predetermined based at least in part on individual user physiological conditions or third party data, or a combination thereof.

In some embodiments, the received user input data may be associated with a value within a numerical range.

In some embodiments, the method may further include receiving third party data corresponding to the one or more subjective user state parameters, and scaling the numerical range of the received user input data based at least in part on the received third party data.

In some embodiments, the one or more physiological parameters may include an at-rest heart rate measured when the user is in a reclined position, an at-rest heart rate measured when the user is in a standing position, a change in at-rest heart rate between when the user is in a reclined position and when the user is in a standing position, an at-rest heart rate variation, orthostatic hypotension, or an intensity of activity, or a combination thereof.

In some embodiments, measuring the at-rest heart rate measured when the user is in a reclined position may include initiating and incrementing an at-rest timer when the user is in the reclined position, and measuring the user's heart rate when the at-rest timer has met or exceeded a first predetermined at-rest threshold.

In some embodiments, measuring the at-rest heart rate measured when the user is in a standing position may include initiating and incrementing the at-rest timer when the user has transitioned from the reclined position to the standing position, and measuring the user's heart rate when the at-rest timer has met or exceeded a second predetermined at-rest threshold.

In some embodiment, the one or more subjective user state parameters may include a value within a numerical range assigned to an average training load over a predetermined period of time, an average training intensity over the predetermined period of time, a quality of sleep, an overall level of life stress, a current level of stress, a quality of food consumed over the predetermined period of time, a quantity of food consumed over the predetermined period of time, a level of pain, or a level of hydration, or a combination thereof.

In some embodiments, the predetermined period of time may be the previous seven days.

In some embodiments, the method may further include receiving recorded data corresponding to orthostatic hypotension for the user, and replacing the received user input data corresponding to the level of hydration with the received recorded data corresponding to orthostatic hypotension for the user.

The present disclosure is also related to a system for determining a readiness of a user. In some embodiments, the system may include a processor configured to: receive physiological data corresponding to one or more physiological parameters of the user; receive user input data corresponding to one or more subjective user state parameters of the user; assign a component score to each of the received physiological data and user input data; assign a respective weight to at least one of the one or more physiological parameters or one or more subjective user state parameters of the user; and derive a readiness score for the user based at least in part on the component scores and the corresponding weights. In some embodiments, the system may further include a transceiver configured to communicate the derived readiness score to a display device.

The present disclosure is also related to a non-transitory computer-readable medium storing computer-executable code. In some embodiments, the code may be executable by the processor to: receive physiological data corresponding to one or more physiological parameters of the user; receive user input data corresponding to one or more subjective user state parameters of the user; assign a component score to each of the received physiological data and user input data; assign a respective weight to at least one of the one or more physiological parameters or one or more subjective user state parameters of the user; derive a readiness score for the user based at least in part on the component scores and the corresponding weights; and communicate the derived readiness score to the user via a display device.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Further scope of the applicability of the described methods and apparatuses will become apparent from the following detailed description, claims, and drawings. The detailed description and specific examples are given by way of illustration only, since various changes and modifications within the spirit and scope of the description will become apparent to those skilled in the art.

### BRIEF DESCRIPTION OF THE DRAWINGS

A further understanding of the nature and advantages of the present invention may be realized by reference to the following drawings. In the appended figures, similar components or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIG. 1 is a block diagram of an example of a physiological parameter monitoring system in accordance with various embodiments;

FIG. 2 is a block diagram of an example of an apparatus in accordance with various embodiments;

FIG. 3 is a block diagram of an example of an apparatus in accordance with various embodiments;

FIG. 4 is a block diagram of an example of an apparatus in accordance with various embodiments;

FIG. 5 is a block diagram of an example of a server for facilitating determining a readiness of a user in accordance with various embodiments;

FIG. 6 is a flowchart of a method for determining a readiness of a user, in accordance with various embodiments;

FIG. 7 is a flowchart of a method for determining a reclining at-rest heart rate for a user;

FIG. 8 is a flowchart of a method for determining a standing at-rest heart rate for a user;

FIG. 9 is an example user interface for inputting data corresponding to one or more subjective user state parameters of the user, in accordance with various embodiments; and

FIG. 10 is a graph displaying physiological parameters of a user received when the user is at-rest in reclined and standing positions, in accordance with various embodiments.

### DETAILED DESCRIPTION

In order to efficiently understand the physiological condition of a user, clinicians may regularly monitor a plurality of physiological parameters of the user. These parameters may include, for example, the user's heart rate, blood pressure, oxygen saturation levels, glucose levels, weight, etc. The different physiological parameters, however, may be incomplete in providing a user's overall health status. Thus, it may be desirable to incorporate data relating to subjective user state parameters, such as quality of sleep or food consumed, in order to present a more holistic view of the user's physical status or “readiness” for any given physical endeavor. Presenting this data to the user or an interested third party, such as a physician or trainer, may be particularly helpful when provided in the form of a numerical score, such that the “readiness” of the user may be compared with that of other users or with the monitored user's own previous scores.

For example, the user may desire to know his health status or readiness before he begins his day, or prior to engaging in a physical activity such as a sports game. The user may benefit from being presented with a single score, which has taken into account both the user's relevant physiological parameters, as well as his subjective user state parameters. Importantly, the physiological parameters and subjective user state parameters may be assigned respective weights commensurate with the impact of each parameter on the user's overall health. The present disclosure includes a method and system for determining a readiness of a user using these detected parameters and their respective weights.

In some examples, deriving the readiness score for the user may be performed at predetermined intervals, for example every morning upon the user's awakening. In other examples, the readiness score may be derived continuously over a predetermined period of time, for example over the course of a day or a training session. In still other examples, the readiness score may be derived by calculating a weighted average of the respective component scores for each of the received physiological data and user input data over a predetermined period of time, where the respective component scores are weighted by their respective weights.

Monitored physiological parameters may include an at-rest heart rate measured when the user is reclined, an at-rest heart rate measured when the user is in a standing position, a change in at-rest heart rate between when the user is reclined and when the user is in a standing position, or an at-rest heart rate variation, or a combination thereof. Physiological parameters may be detected by one or more sensor units worn or carried by, or otherwise associated with, the user. Received physiological data may then be assigned a respective component score, for example in the range from zero to ten, where zero may be a baseline. Physiological parameter baselines may be predetermined based on individual user physiological parameters, health or training goals, or third-party user averages. For example, users in similar age ranges, body weight ranges, or the like may be assigned similar resting heart rate baseline values.

Monitored subjective user state parameters may include an average training load over a predetermined period of time, an average training intensity over the predetermined period of time, a quality of sleep, an overall level of life stress, a current level of stress, a quality of food consumed over the predetermined period of time, a quantity of food consumed over the predetermined period of time, a level of pain, or a level of hydration, or a combination thereof. Subjective user state parameters may be recorded by receiving user inputted data. For example, a user may answer a readiness survey upon awakening in the morning, which may include a series of subjective user state parameters and corresponding sliding scales relating to the user's current or average state over a predetermined period of time. For example, a user may input his average workout load over the previous seven days by providing an input in the range from “none” to “max.” The sliding scale may be predetermined based on individual user physiological parameters, training or health goals, or third-party averages. Similarly, the user may input his overall life stress by providing an input in the range from “no stress” to “overstressed.” The user inputted data may then be assigned a component score, for example in the range from zero to ten.

In some examples, user inputted data corresponding to one or more subjective user state may be replaced with received recorded data corresponding to the one or more subjective user state. For example, in lieu of receiving user input relating to the user's current level of hydration, orthostatic hypotension data may instead be received from one or more sensor units operable to detect blood pressure, or the like. A readiness score for the user may accordingly be calculated using the received sensor data, rather than, or in some embodiments in addition to, the received user input.

The recorded physiological data may be collected manually or through a physiological monitoring system. One example of a physiological monitoring system is a remote physiological monitoring system. Examples below describe such a system, though it should be understood that any type of physiological monitoring system may provide data streams from which the most physiologically relevant parameter values may be selected for display to a clinician. The time period between displayed data values may be adjusted by the clinician in order to best present the data that is most meaningful to the clinician.

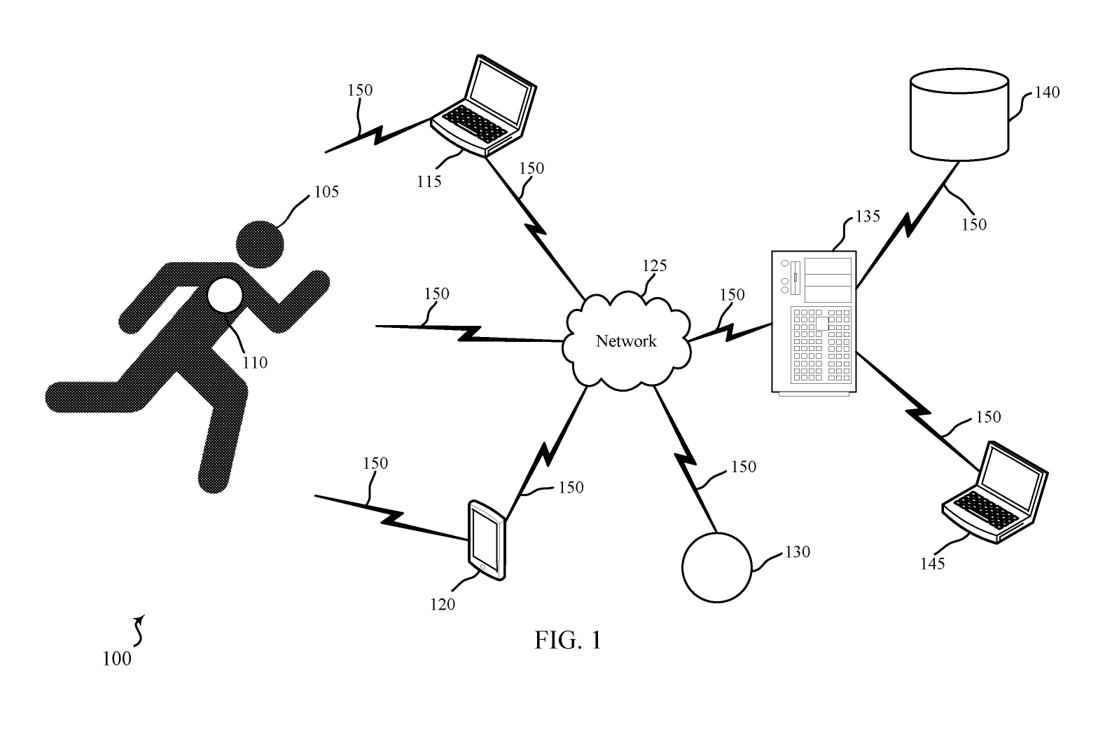

Referring first to FIG. 1, a diagram illustrates an example of a physiological parameter monitoring system 100. The system 100 includes user 105, wearing, carrying, or otherwise associated with one or more sensor unit 110. The user 105 may be an athlete in some examples, may be a patient in other examples, or in some instances may be a layperson interested in simply monitoring his or her health status. The sensor units 110 may transmit signals via wireless communication links 150. The transmitted signals may be transmitted to local computing devices 115, 120. Local computing device 115 may be a local caregiver's station or a personal computing device monitored by a coach, for example. Local computing device 120 may be a mobile device, for example. The local computing devices 115, 120 may be in communication with a server 135 via network 125. The sensor units 110 may also communicate directly with the server 135 via the network 125. Additional, third-party sensors 130 may also communicate directly with the server 135 via the network 125. The server 135 may be in further communication with a remote computing device 145, thus allowing a caregiver or coach to remotely monitor the user 105. The server 135 may also be in communication with various remote databases 140 where the collected data may be stored.

The sensor units 110 are described in greater detail below. Each sensor unit 110 may be capable of sensing multiple physiological parameters. Thus, the sensor units 110 may each include multiple sensors such as heart rate and ECG sensors, respiratory rate sensors, accelerometers, and global positioning sensors. For example, a first sensor in a sensor unit 110 may be an oxygen saturation monitor or a glucose level monitor operable to detect a user's blood oxygen or sugar levels. A second sensor within a sensor unit 110 may be operable to detect a second physiological parameter. For example, the second sensor may be a heart rate monitor, an electrocardiogram (ECG) sensing module, a breathing rate sensing module, and/or any other suitable module for monitoring any suitable physiological parameter. Multiple sensor units 110 may be used on a single user 105. The sensor units 110 may be worn or carried by the user 105 through any known means, for example as a wearable chest strap or wristwatch-type device, or the like. In other examples, the sensor units 110 may be integrated with the user's clothing. The data collected by the sensor units 110 may be wirelessly conveyed to either the local computing devices 115, 120 or to the remote computing device 145 (via the network 125 and server 135). Data transmission may occur via, for example, frequencies appropriate for a personal area network (such as Bluetooth or IR communications) or local or wide area network frequencies such as radio frequencies specified by the IEEE 802.15.4 standard.

Each data point recorded by the sensor units 110 may include an indication of the time the measurement was made (referred to herein as a “timestamp”). In some embodiments, the sensor units 110 are sensors configured to conduct periodic automatic measurements of one or more physiological parameters. A user may wear or otherwise be attached to one or more sensor units 110 so that the sensor units 110 may measure, record, and/or report physiological data associated with the user.

The sensor units 110 may be discrete sensors, each having independent clocks. As a result, sensor units 110 may generate data with different frequencies. The data streams generated by the sensor units 110 may also be offset from each other. The sensor units 110 may each generate a data point at any suitable time interval.

The local computing devices 115, 120 may enable the user 105 and/or a local caregiver or coach to monitor the collected physiological data. For example, the local computing devices 115, 120 may be operable to present data collected from sensor units 110 in a human-readable format. For example, the received data may be outputted as a display on a computer or a mobile device. The local computing devices 115, 120 may include a processor that may be operable to present data received from the sensor units 110 in a visual format. The local computing devices 115, 120 may also output data in an audible format using, for example, a speaker.

The local computing devices 115, 120 may additionally be operable to receive user inputted data corresponding to one or more subjective physiological data parameters of the user. For example, the user may input at a smartphone or personal computing device details related to his quality of sleep, level of stress, consumption of food, and the like. The inputted user data may be associated with a particular time period, such as the current or previous day, or the previous week. The local computing devices 115, 120 may combine the inputted user data with received physiological data, and may derive a readiness score therefrom, as discussed in more detail below.

The local computing devices 115, 120 may be custom computing entities configured to interact with the sensor units 110 and receive user input. In some embodiments, the local computing devices 115, 120 and the sensor units 110 may be portions of a single sensing unit operable to sense and display physiological parameters, for example on a wrist-worn monitor. In another embodiment, the local computing devices 115, 120 may be general purpose computing entities such as a personal computing device, for example, a desktop computer, a laptop computer, a netbook, a tablet personal computer (PC), an iPod®, an iPad®, a smartphone (e.g., an iPhone®, an Android® phone, a Blackberry®, a Windows® phone, etc.), a mobile phone, a personal digital assistant (PDA), and/or any other suitable device operable to send and receive signals, store and retrieve data, and/or execute modules.

The local computing devices 115, 120 may include memory, a processor, an output, a data input, and a communication module. The processor may be a general purpose processor, a Field Programmable Gate Array (FPGA), an Application Specific Integrated Circuit (ASIC), a Digital Signal Processor (DSP), and/or the like. The processor may be configured to retrieve data from and/or write data to the memory. The memory may be, for example, a random access memory (RAM), a memory buffer, a hard drive, a database, an erasable programmable read only memory (EPROM), an electrically erasable programmable read only memory (EEPROM), a read only memory (ROM), a flash memory, a hard disk, a floppy disk, cloud storage, and/or so forth. In some embodiments, the local computing devices 115, 120 may include one or more hardware-based modules (e.g., DSP, FPGA, ASIC) and/or software-based modules (e.g., a module of computer code stored at the memory and executed at the processor, a set of processor-readable instructions that may be stored at the memory and executed at the processor) associated with executing an application, such as, for example, receiving and displaying data from sensor units 110.

The data input module of the local computing devices 115, 120 may be used to manually input measured physiological data and subjective user state data instead of or in addition to receiving data from the sensor units 110. For example, a third-party user of the local computing device 115, 120 may make an observation as to one or more physiological or subjective conditions of a monitored user and record the observation using the data input module. A third-party user may be, for example, a nurse, a doctor, a coach, and/or any other medical healthcare or physical training professional authorized to record user observations, the monitored user, and/or any other suitable user. For instance, the third-party user may measure the monitored user's body temperature (e.g., using a stand-alone thermometer) and enter the measurement into the data input module. In some embodiments, the data input module may be operable to allow the third-party user to select “body temperature” and input the observed temperature into the data input module, e.g., using a keyboard. The data input module may timestamp the observation (or measurement) with the time the observation is inputted into the local computing devices 115, 120, or the local computing devices 115, 120 may prompt the third-party user to input the time at which the observation (or measurement) was made so that the time provided by the third-party user is used to timestamp the data point. In another example, a third-party user may observe the current stress level of the user, for example on a sliding scale from “no stress” to “overstressed,” and may input corresponding subjective user state parameter observations into the local computing devices 115, 120.

The processor of the local computing devices 115, 120 may be operated to control operation of the output of the local computing devices 115, 120. The output may be a television, liquid crystal display (LCD) monitor, cathode ray tube (CRT) monitor, speaker, tactile output device, and/or the like. In some embodiments, the output may be an integral component of the local computing devices 115, 120. Similarly stated, the output may be directly coupled to the processor. For example, the output may be the integral display of a tablet and/or smartphone. In some embodiments, an output module may include, for example, a High Definition Multimedia Interface™ (HDMI) connector, a Video Graphics Array (VGA) connector, a Universal Serial Bus™ (USB) connector, a tip, ring, sleeve (TRS) connector, and/or any other suitable connector operable to couple the local computing devices 115, 120 to the output.

As described in additional detail herein, at least one of the sensor units 110 may be operable to transmit physiological data to the local computing devices 115, 120 and/or to the remote computing device 145 continuously, at scheduled intervals, when requested, and/or when certain conditions are satisfied (e.g., during an alarm condition).

The remote computing device 145 may be a computing entity operable to enable a remote user to monitor the output of the sensor units 110. The remote computing device 145 may be functionally and/or structurally similar to the local computing devices 115, 120 and may be operable to receive data streams from and/or send signals to at least one of the sensor units 110 via the network 125. The network 125 may be the Internet, an intranet, a personal area network, a local area network (LAN), a wide area network (WAN), a virtual network, a telecommunications network implemented as a wired network and/or wireless network, etc. The remote computing device 145 may receive and/or send signals over the network 125 via communication links 150 and server 135.

The remote computing device 145 may be used by, for example, a healthcare professional or sports coach to monitor the output of the sensor units 110. In some embodiments, as described in further detail herein, the remote computing device 145 may receive an indication of physiological data when the sensors detect an alert condition, when the healthcare provider or coach requests the information, at scheduled intervals, and/or at the request of the healthcare provider, coach, and/or the user 105. For example, the remote computing device 145 may be operable to receive summarized physiological data and user input data from the server 135 and derive a readiness score for the user therefrom. The remote computing device 145 may be located, for example, at a nurses' station or in a user's room in some examples, or in other instances may be located at a personal computing device monitored by a coach or other professional, and may be configured to provide a visual display or summary of the user's physiological state and associated readiness score. In some instances, the local computing devices 115, 120 may also be operable to receive and display physiological data in much the same way that the remote computing device 145 is operable.

The server 135 may be configured to communicate with the sensor units 110, the local computing devices 115, 120, the third-party sensors 130, the remote computing device 145, and databases 140. The server 135 may perform additional processing on signals received from the sensor units 110, local computing devices 115, 120, or third-party sensors 130, or may simply forward the received information to the remote computing device 145 and databases 140. The databases 140 may be examples of electronic health records (“EHRs”) and/or personal health records (“PHRs”), and may be provided by various service providers. The third-party sensor 130 may be a sensor that is not attached to the user 105 but that still provides physiological data that may be useful in connection with the data provided by sensor units 110. In other examples, the third-party sensor 130 may be worn or carried by, or otherwise associated with, a third-party user, and data therefrom may be used for comparison purposes with data collected from the user 105. In certain embodiments, the server 135 may be combined with one or more of the local computing devices 115, 120 and/or the remote computing device 145.

The server 135 may be a computing device operable to receive data streams (e.g., from the sensor units 110 and/or the local computing devices 115, 120), store and/or process data, and/or transmit data and/or data summaries (e.g., to the remote computing device 145). For example, the server 135 may receive a stream of heart rate data from a sensor unit 110, a stream of user posture data from the same or a different sensor unit 110, and a stream of user input data from a local computing device 115, 120. In some embodiments, the server 135 may “pull” the data streams, e.g., by querying the sensor units 110 and/or the local computing devices 115, 120. In some embodiments, the data streams may be “pushed” from the sensor units 110 and/or the local computing devices 115, 120 to the server 135. For example, the sensor units 110 and/or the local computing devices 115, 120 may be configured to transmit data as it is generated by or entered into that device. In some instances, the sensor units 110 and/or the local computing devices 115, 120 may periodically transmit data (e.g., as a block of data or as one or more data points).

The server 135 may include a database (e.g., in memory) containing physiological data received from the sensor units 110 and/or the local computing devices 115, 120. The server 135 may additionally contain data associated with subjective user state parameters and parameters associated with third parties. Additionally, as described in further detail herein, software (e.g., stored in memory) may be executed on a processor of the server 135. Such software (executed on the processor) may be operable to cause the server 135 to monitor, process, summarize, present, and/or send a signal associated with physiological data and/or subjective user state parameters.

Although the server 135 and the remote computing device 145 are shown and described as separate computing devices, in some embodiments, the remote computing device 145 may perform the functions of the server 135 such that a separate server 135 may not be necessary. In such an embodiment, the remote computing device 145 may receive physiological data streams from the sensor units 110 and/or the local computing devices 115, 120, process the received data, and derive a user readiness score therefrom.

Additionally, although the remote computing device 145 and the local computing devices 115, 120 are shown and described as separate computing devices, in some embodiments, the remote computing device 145 may perform the functions of the local computing devices 115, 120 such that a separate local computing device 115, 120 may not be necessary. In such an embodiment, the third-party user (e.g., a nurse or a coach) may manually enter the user's physiological and/or subjective user state parameter data (e.g., the user's body temperature, pain level, etc.) directly into the remote computing device 145.

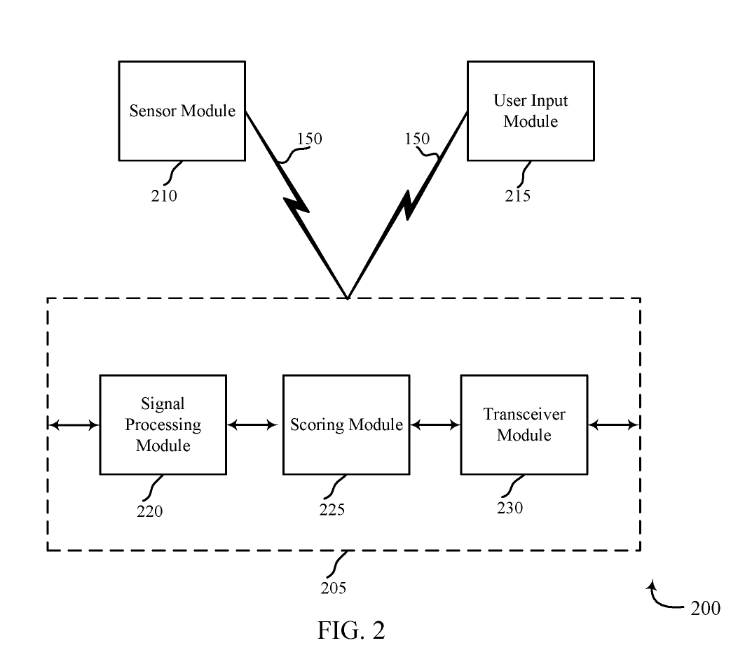

FIG. 2 shows a block diagram 200 that includes apparatus 205, which may be an example of one or more aspects of the sensor unit 110, third-party sensor 130, local computing devices 115, 120, and/or remote computing device 145 (of FIG. 1) for use in physiological monitoring, in accordance with various aspects of the present disclosure. In some examples, the apparatus 205 may include a signal processing module 220, a scoring module 225, and a transceiver module 230. In some examples, one or more sensor modules 210 and/or user input modules 215 may be positioned externally to apparatus 205 and may communicate with apparatus 205 via wireless links 150, or in other examples the one or more sensor modules 210 and/or user input modules 215 may be components of apparatus 205. Each of these components may be in communication with each other.

The components of the apparatus 205 may, individually or collectively, be implemented using one or more application-specific integrated circuits (ASICs) adapted to perform some or all of the applicable functions in hardware. Alternatively, the functions may be performed by one or more other processing units (or cores), on one or more integrated circuits. In other examples, other types of integrated circuits may be used (e.g., Structured/Platform ASICs, Field Programmable Gate Arrays (FPGAs), and other Semi-Custom ICs), which may be programmed in any manner known in the art. The functions of each unit may also be implemented, in whole or in part, with instructions embodied in a memory, formatted to be executed by one or more general or application-specific processors.

In some examples, the signal processing module 220 may include circuitry, logic, hardware and/or software for processing the data streams received from the sensor units 110, sensor modules 210, and/or user input modules 215. The signal processing module 220 may include filters, analog-to-digital converters and other digital signal processing units. Data processed by the signal processing module 220 may be stored in a buffer, for example.

Data streams processed by signal processing module 220 may then be communicated to scoring module 225. Scoring module 225 may be operable to assign a respective component score to each of the received physiological data and user input data streams, and to assign a respective weight to at least one of the one or more physiological parameters or one or more subjective user state parameters of the user. The weighted data may then be communicated to the transceiver module 230 for further processing and transmission.

In some examples, the transceiver module 230 may be operable to receive data streams from the scoring module 225, as well as to send and/or receive other signals between the sensor units 110 and either the local computing devices 115, 120 or the remote computing device 145 via the network 125 and server 135. In an embodiment, the transceiver module 230 may receive data streams from the scoring module 225 and may also forward the data streams to other devices. The transceiver module 230 may include wired and/or wireless connectors. For example, in some embodiments, sensor units 110 may be portions of a wired or wireless sensor network, and may communicate with the local computing devices 115, 120 and/or remote computing device 145 using either a wired or wireless network. The transceiver module 230 may be a wireless network interface controller (“NIC”), Bluetooth® controller, IR communication controller, ZigBee® controller and/or the like. Transceiver module 230 may also be operable to derive a readiness score for the user based at least in part on the component scores and corresponding weights of the data received from scoring module 225. Transceiver module 230 may then communicate the derived readiness score to the user at, for example, a local computing device 115, 120 or remote computing device 145.

Sensor module 210 may comprise any combination of physiological sensing components, including, for example, heart rate monitors, respiration monitors, blood pressure monitors, pulse monitors, orientation monitors, accelerometers, temperature monitors, global positioning sensors, force monitors, and the like. User input module 215 may comprise any control panel, personal computing device, dedicated application, or the like, operable to receive user input related to one or more subjective user state parameters of the user. For example, the user may input data related to any of workout load, workout intensity, sleep quality, overall life stress, current day's stress, quality of food, quantity of food, pain level, and hydration level, or the like. A user may input subjective user state parameter data in the form of a numerical representation in some examples, or may adjust a sliding scale in other examples.

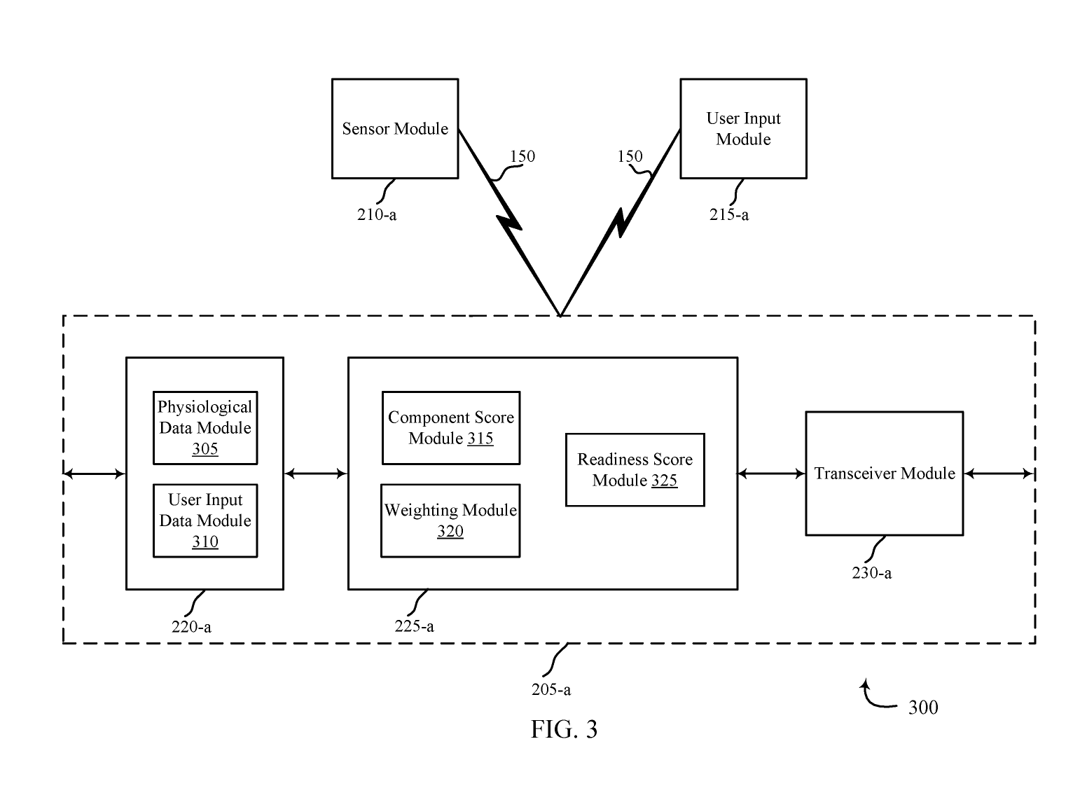

FIG. 3 shows a block diagram 300 that includes apparatus 205-a, which may be an example of apparatus 205 (of FIG. 2), in accordance with various aspects of the present disclosure. In some examples, the apparatus 205-a may include a signal processing module 220-a, a scoring module 225-a, and a transceiver module 230-a, any of which may be examples of the signal processing module 220, the scoring module 225, and the transceiver module 230 of FIG. 2. In addition, one or more sensor module 210-a and/or user input module 215-a may be in communication with or integrated with the apparatus 205-a, and may be examples of the sensor module 210 and user input module 215 of FIG. 2. In some examples, signal processing module 220-a may include one or more of a physiological data module 305 and a user input data module 310. In some examples, scoring module 225 may include one or more of a component score module 315, a weighting module 320, or a readiness score module 325. Additionally, while FIG. 3 illustrates a specific example, the functions performed by each of the modules 305, 310, 315, 320, and 325 may be combined or implemented in one or more other modules.

The physiological data module 305 may be operable to receive physiological data corresponding to one or more physiological parameters of the monitored user. Physiological data may be monitored by one or more sensor units, sensor module 210-a, or may be inputted by a third-party user at a local or remote computing device. Monitored physiological parameters may include an at-rest heart rate measured when the user is in a reclined position, an at-rest heart rate measured when the user is in a standing position, a change in at-rest heart rate between when the user is in a reclined position and when the user is in a standing position, an at-rest heart rate variation, orthostatic hypertension, intensity of physical activity, and the like. In some examples, physiological data module 305 may receive physiological data and derive a physiological parameter therefrom. In other examples, physiological data module 305 may communicate the received physiological data to scoring module 225-a without prior processing.

User input data module 310 may similarly be operable to receive user input data corresponding to one or more subjective user state parameters of the monitored user. User input data may be received at user input module 215-a, or may be inputted at a local or remote computing device. For example, a user may respond to a survey on a dedicated application on his smartphone. Subjective user state parameters may include average training load over a predetermined period of time, average training intensity over the predetermined period of time, quality of sleep, overall level of life stress, current level of stress, quality of food consumed over the predetermined period of time, quantity of food consumed over the predetermined period of time, level of pain, and/or level of hydration. In some examples, user input data module 310 may receive user input data and derive a subjective user state parameter therefrom. In other examples, user input data module 310 may communicate the received user input data to scoring module 225-a without prior processing.

Component score module 315 may be operable to receive physiological data from physiological data module 305, and user input data from user input data module 310, and assign a respective component score to each of the received physiological data and user input data. The respective component scores may in some examples include a numerical range of 0-10.

Weighting module 320 may be operable to provide a respective, predetermined weighting to each of the monitored physiological and subjective user state parameters monitored. Respective recommended weightings may be predetermined based on individual user physiological parameters, or may be based on third party physiological parameters or averages. In other examples, a user may provide input directed to desired weightings according to personal physical or training preferences. For example, a resting heart rate measured for a reclined user may be assigned a weight of 10, while a training intensity for the user may be assigned a weight of 4, and a quantity of food consumed by the user over the past week may be assigned a weight of 2. Various combinations of weights may be assigned based on individual user parameters or training goals. In some examples, the respective weights may be assigned from a numerical range of 1-10, while in other examples, other numerical ranges may be used.

Readiness score module 325 may collect the respective component scores received from component score module 315, and the respective weights received from weighting module 320, and may derive a readiness score for the user according to the following equation:

$$Readiness Score = \frac{{\sum }_{n = 1}^{13} {Input}_{n} * {Weight}_{n}}{{\sum }_{n = 1}^{13} 10 * {Weight}_{n}}$$

where the sum of all available component scores multiplied by a respective weighting, then divided by the maximum weighted score, may make up the readiness score. Where a certain physiological or subjective user state parameter has not been measured or is not available, the component score for that parameter will not be included in the readiness score calculation, such that the readiness score may still be determined based on partially complete data.

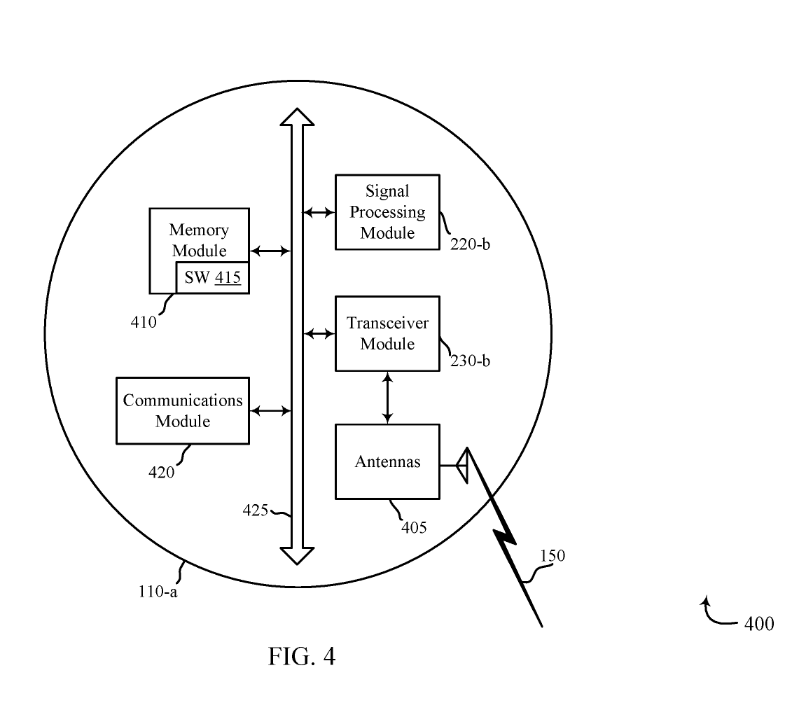

FIG. 4 shows a block diagram 400 of a sensor unit 110-a for use in remote physiological data monitoring, in accordance with various aspects of the present disclosure. The sensor unit 110-a may have various configurations. The sensor unit 110-a may, in some examples, have an internal power supply (not shown), such as a small battery, to facilitate mobile operation. In some examples, the sensor unit 110-a may be an example of one or more aspects of one of the sensor units 110 and/or apparatus 205, 205-a described with reference to FIGS. 1, 2 and/or 3. In some examples, the sensor unit 110-a may be an example of one or more aspects of one of the sensor modules 210, 210-a described with reference to FIGS. 2 and/or 3. The sensor unit 110-a may be configured to implement at least some of the features and functions described with reference to FIGS. 1, 2 and/or 3.

The sensor unit 110-a may include a signal processing module 220-b, a transceiver module 230-b, a communications module 420, at least one antenna (represented by antennas 405), and/or a memory module 410. Each of these components may be in communication with each other, directly or indirectly, over one or more buses 425. The signal processing module 220-b and transceiver module 230-b may be examples of the signal processing module 220 and transceiver module 230, respectively, of FIG. 2.

The memory module 410 may include RAM and/or ROM. The memory module 410 may store computer-readable, computer-executable code (SW) 415 containing instructions that are configured to, when executed, cause the signal processing module 220-b to perform various functions described herein related to deriving a readiness score for a user. Alternatively, the code 415 may not be directly executable by the signal processing module 220-b but may be configured to cause the server 135 (of FIG. 1) (e.g., when compiled and executed) to perform various of the functions described herein.

The signal processing module 220-b may include an intelligent hardware device, e.g., a CPU, a microcontroller, an ASIC, etc. The signal processing module 220-b may process information received through the transceiver module 230-b or information to be sent to the transceiver module 230-b for transmission through the antenna 405. The signal processing module 220-b may handle various aspects of signal processing as well as deriving a readiness score for a user.

The transceiver module 230-b may include a modem configured to modulate packets and provide the modulated packets to the antennas 405 for transmission, and to demodulate packets received from the antennas 405. The transceiver module 230-b may, in some examples, be implemented as one or more transmitter modules and one or more separate receiver modules. The transceiver module 230-b may support readiness score communications. The transceiver module 230-b may be configured to communicate bi-directionally, via the antennas 405 and communication link 150, with, for example, local computing devices 115, 120 and/or the remote computing device 145 (via network 125 and server 135 of FIG. 1). Communications through the transceiver module 230-b may be coordinated, at least in part, by the communications module 420. While the sensor unit 110-a may include a single antenna 405, there may be examples in which the sensor unit 110-a may include multiple antennas 405.

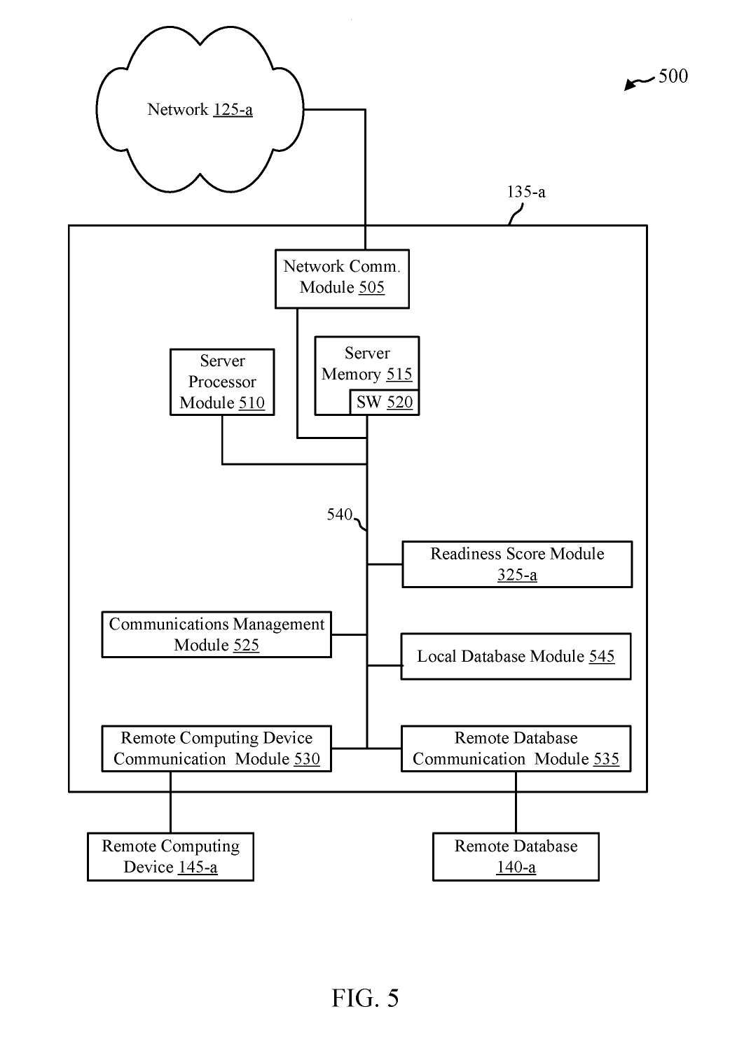

FIG. 5 shows a block diagram 500 of a server 135-a for use in deriving a readiness score for a user, in accordance with various aspects of the present disclosure. In some examples, the server 135-a may be an example of aspects of the server 135 described with reference to FIG. 1. In other examples, the server 135-a may be implemented in either the local computing devices 115, 120 or the remote computing device 145 of FIG. 1. The server 135-a may be configured to implement or facilitate at least some of the features and functions described with reference to the server 135, the local computing devices 115, 120 and/or the remote computing device 145 of FIG. 1.

The server 135-a may include a server processor module 510, a server memory module 515, a local database module 545, and/or a communications management module 525. The server 135-a may also include one or more of a network communication module 505, a remote computing device communication module 530, and/or a remote database communication module 535. Each of these components may be in communication with each other, directly or indirectly, over one or more buses 540.

The server memory module 515 may include RAM and/or ROM. The server memory module 515 may store computer-readable, computer-executable code (SW) 520 containing instructions that are configured to, when executed, cause the server processor module 510 to perform various functions described herein related to determining a readiness score for a user. Alternatively, the code 520 may not be directly executable by the server processor module 510 but may be configured to cause the server 135-a (e.g., when compiled and executed) to perform various of the functions described herein.

The server processor module 510 may include an intelligent hardware device, e.g., a central processing unit (CPU), a microcontroller, an ASIC, etc. The server processor module 510 may process information received through the one or more communication modules 505, 530, 535. The server processor module 510 may also process information to be sent to the one or more communication modules 505, 530, 535 for transmission. Communications received at or transmitted from the network communication module 505 may be received from or transmitted to sensor units 110, local computing devices 115, 120, or third-party sensors 130 via network 125-a, which may be an example of the network 125 described in relation to FIG. 1. Communications received at or transmitted from the remote computing device communication module 530 may be received from or transmitted to remote computing device 145-a, which may be an example of the remote computing device 145 described in relation to FIG. 1. Communications received at or transmitted from the remote database communication module 535 may be received from or transmitted to remote database 140-a, which may be an example of the remote database 140 described in relation to FIG. 1. Additionally, a local database may be accessed and stored at the server 135-a. The local database module 545 may be used to access and manage the local database, which may include data received from the sensor units 110, the local computing devices 115, 120, the remote computing devices 145, or the third-party sensors 130 (of FIG. 1).

The server 135-a may also include a readiness score module 325-a, which may be an example of the readiness score module 325 of apparatus 205-a described in relation to FIG. 3. The readiness score module 325-a may perform some or all of the features and functions described in relation to the readiness score module 325, including collecting the respective component scores received from component score module 315, and the respective weights received from weighting module 320, as described in FIG. 3, and deriving a readiness score for the user therefrom.

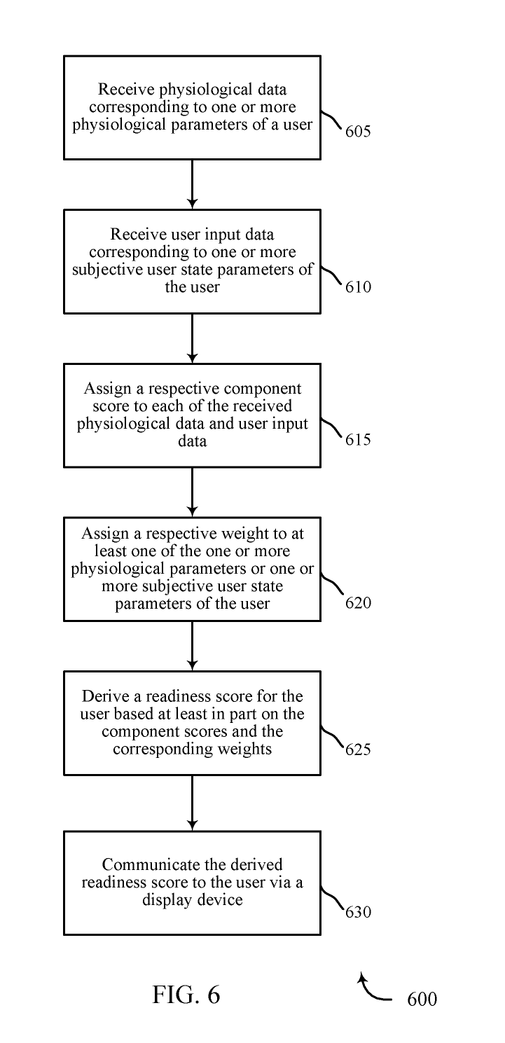

FIG. 6 is a flow chart illustrating an example of a method 600 for determining a readiness of a user, in accordance with various aspects of the present disclosure. For clarity, the method 600 is described below with reference to aspects of one or more of the local computing devices 115, 120, remote computing device 145, and/or server 135 described with reference to FIGS. 1 and/or 5, or aspects of one or more of the apparatus 205, 205-a described with reference to FIGS. 2 and/or 3. In some examples, a local computing device, remote computing device or server such as one of the local computing devices 115, 120, remote computing device 145, server 135 and/or an apparatus such as one of the apparatuses 205, 205-a may execute one or more sets of codes to control the functional elements of the local computing device, remote computing device, server or apparatus to perform the functions described below.

At block 605, the method 600 may include receiving physiological data corresponding to one or more physiological parameters of a user. The physiological data may be collected by one or more sensor units worn or held by, or associated with, the monitored user. The one or more physiological parameters may include, for example, an at-rest heart rate measured when the user is lying down, an at-rest heart rate measured when the user is standing up, a change in at-rest heart rate between when the user is lying down and when the user is standing up, or an at-rest heart rate variation, or a combination thereof.

At block 610, the method 600 may include receiving user input data corresponding to one or more subjective user state parameters of the user. The received user input data may be inputted by the monitored user or a third party user at any of a local or remote computing device. For example, the user may input data relating to one or more subjective user state parameters at a dedicated application on his smartphone or personal computing device. In another example, a third-party user familiar with or observing the monitored user, such as a doctor or coach, may input data corresponding to one or more subjective user state parameters at a website application. The one or more subjective user state parameters may include a value within a numerical range assigned to an average training load over a predetermined period of time, an average training intensity over the predetermined period of time, a quality of sleep, an overall level of life stress, a current level of stress, a quality of food consumed over the predetermined period of time, a quantity of food consumed over the predetermined period of time, a level of pain, or a level of hydration, or a combination thereof.

For example, a user may input data in an online questionnaire every morning on a daily basis. The questionnaire or “readiness survey” may include a list of subjective user state parameters with corresponding user inputs, for example in the form of a series of sliding scales. In one example, input relating to average workout load or intensity may range from “none” to “max,” while input relating to overall life stress may range from “no stress” to “overstressed.” In another example, input relating to average quantity of food may range from “not enough” to “too much,” with a median goal of “just right.” Other combinations and ranges are also envisioned.

The one or more subjective user state parameters may relate to a single predetermined period of time, or may relate to various predetermined periods of time. For example, an input relating to average workout load may relate to the previous seven days, while sleep quality may relate only to the previous night, and current stress level may relate to the present day.

At block 615, the method 600 may include assigning a respective component score to each of the received physiological data and user input data. For example, a baseline may be assigned to an individual physiological parameter or user state parameter based on individual user physiological parameters, training or health goals, or third-party averages. For example, a baseline resting heart rate for a young, physically fit user may be different from a baseline resting heart rate for an elderly, less active user. Variations in individual physiological data with respect to the predetermined baseline may accordingly determine the respective component score for the physiological data. For example, a baseline resting heart rate for a user who is reclined may be 100 beats per minute (bpm), based on an average resting heart rate for third-party users having similar physiological makeups to that of the user. A 100 bpm resting heart rate may accordingly be assigned a component score of zero. A resting heart rate of 20 bpm above the baseline for the reclined user may be assigned a component score often, and any variation between the two may be assigned a component score between one and nine accordingly. Similarly, a scale of 0-10 may be assigned to user input corresponding to subjective user state parameters. For example, user input indicating an unhealthy quality of food over the past seven days may be assigned a component score of 0, while healthy food may be assigned a component score of 10.

Respective component scores may not range from 0-10, where zero represents a negative user state and ten represents a positive user state, in all examples. In some instances, a component score of zero may represent a healthy user state, for example where the user's resting heart rate when in a standing position is at the predetermined baseline. In other examples, zero may represent a negative or unhealthy user state, for example where the user has consumed unhealthy food over the past week. In still other examples, the positive or negative characterization of the component score may vary. For example, where a user is inputting data corresponding to the quantity of food he has consumed over the past seven days, a component score of zero may indicate that he has not eaten enough food, a component score of ten may indicate that he has eaten too much food, and a component score of five may indicate that his food intake was “just right.” User input from zero to five indicating too little to just enough food may then be scaled from zero to ten when calculating overall user readiness, as described in more detail below. Similarly, user input from six to ten indicating just enough to too much food may be similarly scaled from zero to ten.

At block 620, the method 600 may include assigning a respective weight to at least one of the one or more physiological parameters or one or more subjective user state parameters of the user. Respective parameter weightings may be predetermined based on individual user physiological parameters, training or fitness goals, or the like. For example, user resting heart rate may be weighted higher than user training intensity, but lower than resting heart rate variation. In some examples, the physiological parameters may be weighted more heavily than the subjective user state parameters, while in other examples the physiological parameters may be weighted equally to the subjective user state parameters.

At block 625, the method 600 may include deriving a readiness score for the user based at least in part on the component scores and the corresponding weights. For example, the readiness score may be calculated according to the following equation:

$$Readiness Score = \frac{{\sum }_{n = 1}^{13} {Input}_{n} * {Weight}_{n}}{{\sum }_{n = 1}^{13} 10 * {Weight}_{n}}$$

In particular, the sum of all available component scores may be multiplied by a respective weighting, and divided by the maximum weighted score in order to derive the readiness score for the user. Where data related to one or more physiological parameter and/or subjective user state parameter is unavailable at the time of calculation, the missing data may be nulled such that a readiness score may nonetheless be derived for the user. The derivation of the readiness score may be performed at any of the one or more sensor units, local computing devices, or remote computing device, as discussed above with respect to FIGS. 2 and 3.

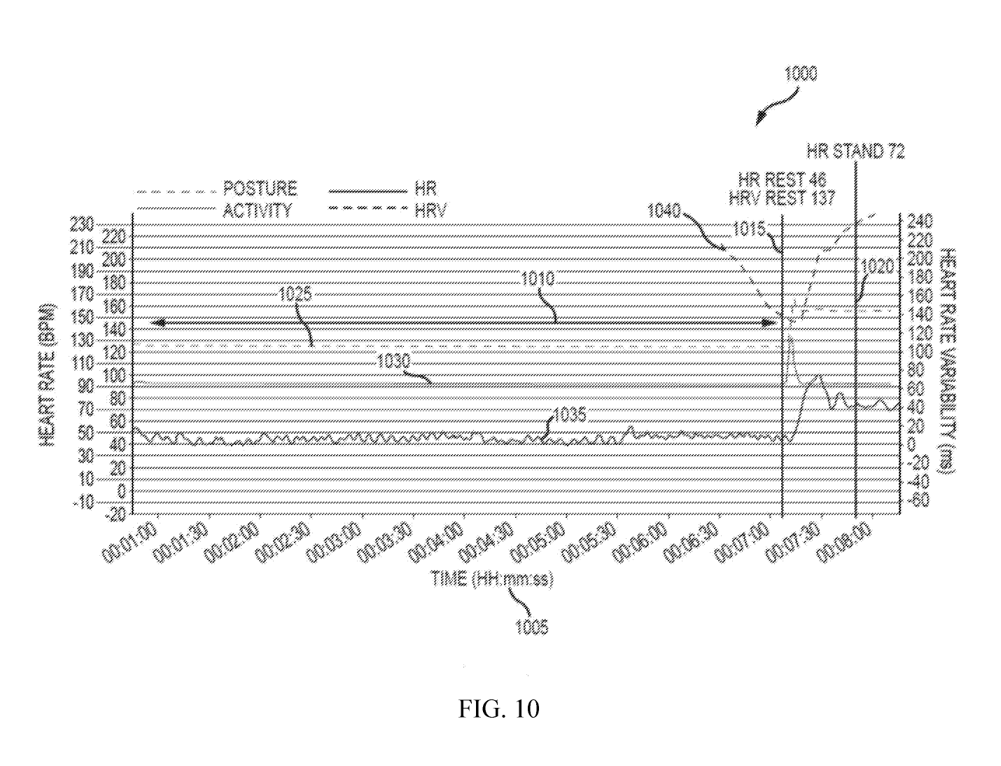

At block 630, the method 600 may include communicating the derived readiness score to the user via a display device. For example, where the readiness score is derived at a sensor unit worn by or otherwise associated with the user, the readiness score may be displayed on a wrist-worn display device, or may alternatively or in addition be communicated to a dedicated application on the user's smartphone or personal computing device. In some examples, the readiness score may be calculated for a single point in time, for example upon detection of data corresponding to one or more physiological parameters and subjective user state parameters upon the user's awakening in the morning. Accordingly, the derived readiness score may be communicated to the user as a single numerical indicator, symbol, or associated color. In other examples, the user's readiness score may be continuously or periodically updated based on incoming data associated with one or more physiological parameters and subjective user state parameters. For example, as the user goes about his day, engages in a physical activity, or the like, updated data corresponding to heart rate, stress level, and the like may be detected and/or inputted, and taken into account in determining an updated readiness score. Accordingly, the readiness score may be delivered to the user as an “over time” graph or other visual representation to indicate the change in user readiness over time, as discussed in more detail below with respect to FIG. 10.

In some embodiments, the operations at blocks 605, 610, 615, 620, or 625 may be performed using the signal processing module 220, scoring module 225, and transceiver module 230 described with reference to FIGS. 2 and 3. Nevertheless, it should be noted that the method 600 is just one implementation and that the operations of the method 600 may be rearranged or otherwise modified such that other implementations are possible.

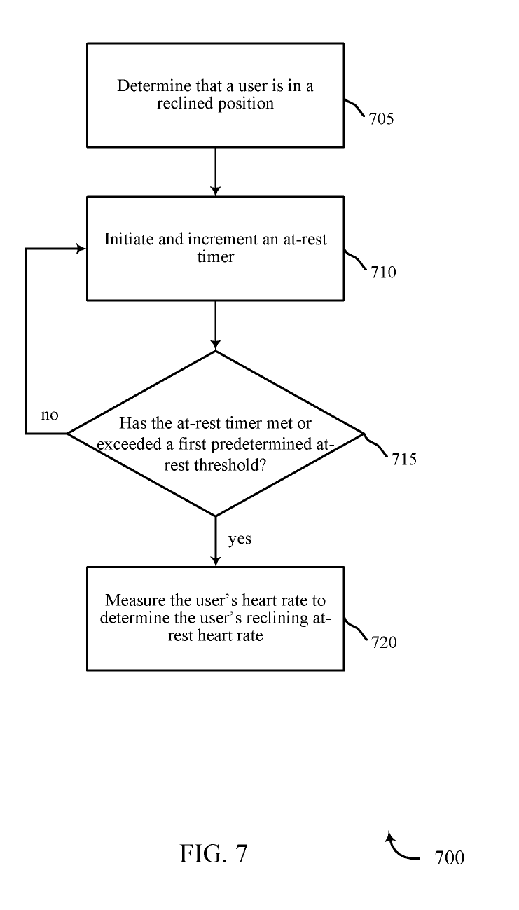

FIG. 7 is a flow chart illustrating an example of a method 700 for determining a reclined resting heart rate for a user, in accordance with various aspects of the present disclosure. For clarity, the method 700 is described below with reference to aspects of one or more of the local computing devices 115, 120, remote computing device 145, and/or server 135 described with reference to FIGS. 1 and/or 5, or aspects of one or more of the apparatus 205, 205-a described with reference to FIGS. 2 and/or 3. In some examples, a local computing device, remote computing device or server such as one of the local computing devices 115, 120, remote computing device 145, server 135 and/or an apparatus such as one of the apparatuses 205, 205-a may execute one or more sets of codes to control the functional elements of the local computing device, remote computing device, server, or apparatus to perform the functions described below.

At block 705, the method 700 may include determining that a user is in a reclined position. In some examples, this determination may be performed by one or more sensor units coupled to or otherwise associated with the user, where the one or more sensor units may include a gyroscope, accelerometer, or other position detection means. In other examples, user posture may be observed and inputted by a third-party user at a local or remote computing device.

At block 710, the method 700 may include initiating and incrementing an at-rest timer. At block 715, the method 700 may include determining whether the at-rest timer has met or exceeded a first predetermined at-rest threshold. The first predetermined at-rest threshold may be determined based at least in part on individual user physiological parameters, training or health goals, or third-party user averages. For example, the at-rest threshold for a young, physically fit user may be less than that for an elderly, less active user.

At block 715, if it is determined that the at-rest timer has met or exceeded the first predetermined at-rest threshold, the method 700 may continue to block 720, in which the user's heart rate may be measured to determine the user's reclining at-rest heart rate. Alternatively, if at block 715 it is determined that the at-rest timer has not met or exceeded the first predetermined at-rest threshold, the method 700 may return to block 710 to continue incrementing the at-rest timer until such time as the at-rest timer has met or exceeded the first predetermined at-rest threshold. In this way, it may be ensured that the user's reclined resting heart rate is measured only when the user is truly at-rest.

The measured reclined at-rest heart rate may then be assigned a component score and weighted score, and may be included in a readiness score calculation, as previously discussed with respect to FIG. 6. In some embodiments, the operations at blocks 705, 710, 715, or 720 may be performed using the signal processing module 220, scoring module 225, and transceiver module 230 described with reference to FIGS. 2 and 3. Nevertheless, it should be noted that the method 700 is just one implementation and that the operations of the method 700 may be rearranged or otherwise modified such that other implementations are possible.

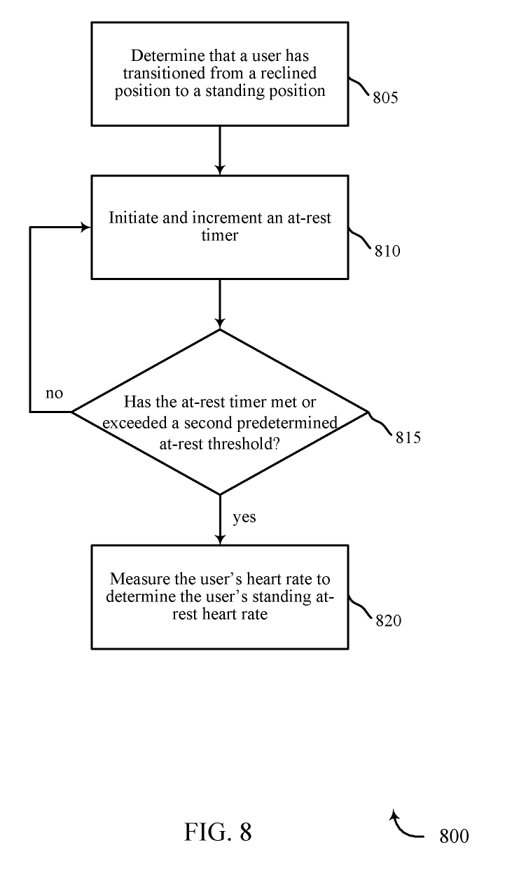

FIG. 8 is a flow chart illustrating an example of a method 800 for determining a standing resting heart rate for a user, in accordance with various aspects of the present disclosure. For clarity, the method 800 is described below with reference to aspects of one or more of the local computing devices 115, 120, remote computing device 145, and/or server 135 described with reference to FIGS. 1 and/or 5, or aspects of one or more of the apparatus 205, 205-a described with reference to FIGS. 2 and/or 3. In some examples, a local computing device, remote computing device, or server such as one of the local computing devices 115, 120, remote computing device 145, server 135 and/or an apparatus such as one of the apparatuses 205, 205-a may execute one or more sets of codes to control the functional elements of the local computing device, remote computing device, server, or apparatus to perform the functions described below.

At block 805, the method 800 may include determining that a user has transitioned from a reclined position to a standing position. In some examples, this determination may be performed by one or more sensor units coupled to or otherwise associated with the user, where the one or more sensor units may include a gyroscope, accelerometer, or other position detection means. In other examples, user posture may be observed and inputted by a third-party user at a local or remote computing device.

At block 810, the method 800 may include initiating and incrementing an at-rest timer. At block 815, the method 800 may include determining whether the at-rest timer has met or exceeded a second predetermined at-rest threshold. The second predetermined at-rest threshold may be determined based at least in part on individual user physiological parameters, training or health goals, or third-party user averages. For example, the at-rest threshold for a young, physically fit user may be less than that for an elderly, less active user. In some examples, the first at-rest threshold may be equal to the second at-rest threshold, while in other examples the two thresholds may be different, for example due to individual user physiological parameters. For example, for a particular user, it may take less time for the user to attain an at-rest status when reclined, but may take more time to attain an at-rest status when in a standing position.

At block 815, if it is determined that the at-rest timer has met or exceeded the second predetermined at-rest threshold, the method 800 may continue to block 820, in which the user's heart rate may be measured to determine the user's standing at-rest heart rate. Alternatively, if at block 815 it is determined that the at-rest timer has not met or exceeded the second predetermined at-rest threshold, the method 800 may return to block 810 to continue incrementing the at-rest timer until such time as the at-rest timer has met or exceeded the second predetermined at-rest threshold. In this way, it may be ensured that the user's standing resting heart rate is measured only when the user is truly at-rest.

The measured standing at-rest heart rate may then be assigned a component score and weighted score, and may be included in a readiness score calculation, as previously discussed with respect to FIG. 6. In some embodiments, the operations at blocks 805, 810, 815, or 820 may be performed using the signal processing module 220, scoring module 225, and transceiver module 230 described with reference to FIGS. 2 and 3. Nevertheless, it should be noted that the method 800 is just one implementation and that the operations of the method 800 may be rearranged or otherwise modified such that other implementations are possible.

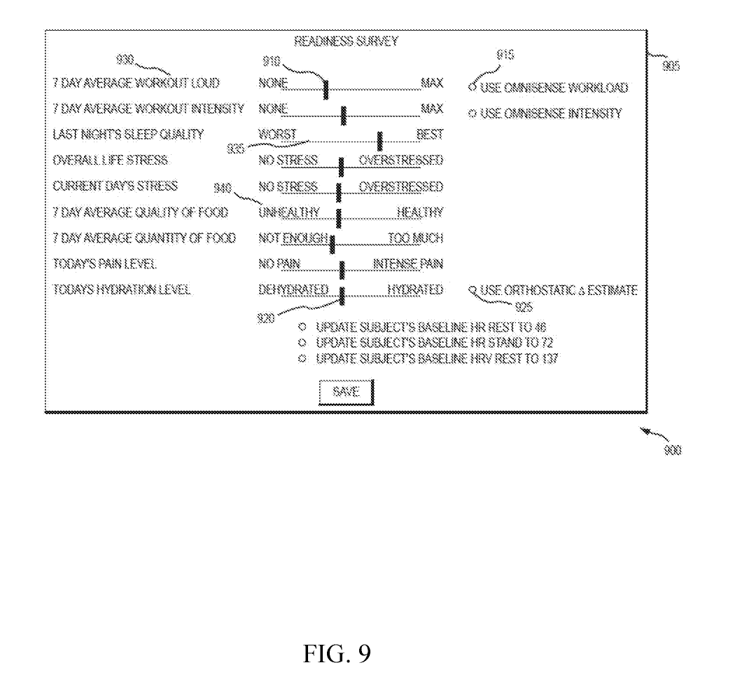

FIG. 9 is an illustration 900 of an example user interface 905 for receiving user input data corresponding to one or more subjective user state parameters 930 of the user. In the illustrated example, the user interface 905 is shown as an interactive readiness survey, viewable for example on a dedicated application on a smartphone, tablet, or personal computer, or in other examples viewable on a webpage from a remote computing device.

The readiness survey may include a plurality of inputs 910, 915, 920, and 925, each associated with user inputted data and/or data gathered from a sensor unit, local or remote computing device, or server. For example, a user may input data relating to his average workout load over the previous seven days using input 910, which may allow for an input ranging from “none” to “max.” The range from “none” to “max” may be a sliding input scale, which may be predetermined and varied based on individual user physiological parameters, health or training goals, or third-party user averages. Other subjective user state parameters, such as last night's sleep quality may be inputted using input 935, in a range of, for example, “worst” to “best”; similarly, input 940 related to a 7 day average quality of food may be inputted in a range from “unhealthy” to “healthy.” In the alternative, the user may select input 915 to indicate that his average workout load over the previous seven days should be pulled from data collected by one or more sensor units associated with the user (e.g., an “Omnisense Workload”). For example, a user wearing a sensor having a heart rate monitor, respiratory monitor, or the like, may elect to have his average workout load inputted automatically based on data monitored by the sensor and stored at, for example, a local or remote computing device or server. Similarly, a user may input data relating to his hydration level for the current day using input 920, which may allow for an input ranging from “dehydrated” to “hydrated.” The range from “dehydrated” to “hydrated” may be a sliding input scale, and may be similarly predetermined and varied based on individual user physiological parameters, health or training goals, or third-party user averages. In the alternative, the user may select input 925 to indicate that his current hydration level should be pulled from data collected by one or more sensor units associated with the user. For example, orthostatic hypotension for a user may be detected using one or more blood pressure sensors or the like, and may be stored at the sensor, or at a local or remote computing device or server. Thus, where the user selects an input 915, 925 to pull data from a sensor or server, the retrieved data may be used in lieu of user inputted data in performing the readiness calculation.

FIG. 10 is an illustration 1000 of an alternate visual representation of user readiness over time. The illustration 1000 may be an interactive user interface, and may be viewable, for example, on a dedicated application on a user's smartphone or personal computer, or any body-worn display device.

In the example shown in illustration 1000, the visual representation is an “over time” graph 1005 to display multiple physiological parameters, such as user posture, activity level, reclined at-rest heart rate, standing at-rest heart rate, heart rate variation, and the like, monitored over time. For example, line 1010 may represent a period of time spent by a user both reclined and having an activity level variation of less than 0.1. Line 1025 may indicate user posture over a period of time, where line 1025 indicates that the user is reclined up until seven minutes, at which time the user moves to a standing position, as shown by line 1015. Similarly, line 1030 may indicate an activity level of the user over the same period of time, showing that the patient is at-rest up until seven minutes, at which time the user becomes active, as also shown by line 1015. The at-rest and/or posture status of the user may be determined based at least in part on one or more sensor units worn or carried by, or otherwise associated with, the user, and operable to measure, for example, acceleration, heart rate, respiration, and the like. At line 1015, the user may move from a reclined, resting position to a standing position. The user's heart rate may be continuously measured over time, as indicated by line 1035 while the user is at rest and reclined. In order to detect an accurate heart rate when the user is standing and at-rest, as discussed in more detail with respect to FIG. 8, a predetermined at-rest threshold may be measured. In the illustrated example, the threshold is about one minute, as shown by line 1020. Upon reaching the at-rest threshold at line 1020, the user's standing at-rest heart rate may be measured, which in this example is detected to be 72 beats per minute (bpm). The heart rate variation between the user's reclining heart rate and standing heart rate may be indicated by line 1040.

The above description provides examples, and is not limiting of the scope, applicability, or configuration set forth in the claims. Changes may be made in the function and arrangement of elements discussed without departing from the spirit and scope of the disclosure. Various embodiments may omit, substitute, or add various procedures or components as appropriate. For instance, the methods described may be performed in an order different from that described, and various steps may be added, omitted, or combined. Also, features described with respect to certain embodiments may be combined in other embodiments.

The detailed description set forth above in connection with the appended drawings describes exemplary embodiments and does not represent the only embodiments that may be implemented or that are within the scope of the claims. The term “exemplary” used throughout this description means “serving as an example, instance, or illustration,” and not “preferred” or “advantageous over other embodiments.” The detailed description includes specific details for the purpose of providing an understanding of the described techniques. These techniques, however, may be practiced without these specific details. In some instances, well-known structures and devices are shown in block diagram form in order to avoid obscuring the concepts of the described embodiments.

Information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, multiple microprocessors, one or more microprocessors in conjunction with a DSP core, or any other such configuration. A processor may in some cases be in electronic communication with a memory, where the memory stores instructions that are executable by the processor.

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope and spirit of the disclosure and appended claims. For example, due to the nature of software, functions described above may be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items indicates a disjunctive list such that, for example, a list of “at least one of A, B, or C” means A or B or C or AB or AC or BC or ABC (i.e., A and B and C).

A computer program product or computer-readable medium both include a computer-readable storage medium and communication medium, including any mediums that facilitates transfer of a computer program from one place to another. A storage medium may be any medium that may be accessed by a general purpose or special purpose computer. By way of example, and not limitation, computer-readable medium may comprise RAM, ROM, EEPROM, CD-ROM or other optical disk storage, magnetic disk storage or other magnetic storage devices, or any other medium that may be used to carry or store desired computer-readable program code in the form of instructions or data structures and that may be accessed by a general-purpose or special-purpose computer, or a general-purpose or special-purpose processor. Also, any connection is properly termed a computer-readable medium. For example, if the software is transmitted from a website, server, or other remote light source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include compact disc (CD), laser disc, optical disc, digital versatile disc (DVD), floppy disk and blu-ray disc where disks usually reproduce data magnetically, while discs reproduce data optically with lasers. Combinations of the above are also included within the scope of computer-readable media.

The previous description of the disclosure is provided to enable a user skilled in the art to make or use the disclosure. Various modifications to the disclosure will be readily apparent to those skilled in the art, and the generic principles defined herein may be applied to other variations without departing from the spirit or scope of the disclosure. Throughout this disclosure the term “example” or “exemplary” indicates an example or instance and does not imply or require any preference for the noted example. Thus, the disclosure is not to be limited to the examples and designs described herein but is to be accorded the widest scope consistent with the principles and novel features disclosed herein.

1. A method for determining a readiness of a user, comprising:
receiving physiological data corresponding to one or more physiological parameters of the user;
receiving user input data corresponding to one or more subjective user state parameters of the user;
assigning a respective component score to each of the received physiological data and user input data;
assigning a respective weight to at least one of the one or more physiological parameters or one or more subjective user state parameters of the user;
deriving a readiness score for the user based at least in part on the component scores and the corresponding weights; and
communicating the derived readiness score to the user via a display device.

2. The method of claim 1, wherein deriving the readiness score further comprises:
calculating a weighted average of the respective component scores for each of the received physiological data and user input data, where the respective component scores are weighted by respective weights.

3. The method of claim 1, further comprising:
receiving recorded data corresponding to the one or more subjective user state parameters of the user; and
replacing the received user input data with the received recorded data.

4. The method of claim 1, wherein the respective component score assigned to each of the received physiological data and user input data is predetermined based at least in part on individual user physiological conditions or third party data, or a combination thereof.

5. The method of claim 1, wherein the received user input data is associated with a value within a numerical range.

6. The method of claim 5, further comprising:
receiving third party data corresponding to the one or more subjective user state parameters; and
scaling the numerical range of the received user input data based at least in part on the received third party data.

7. The method of claim 1, wherein the one or more physiological parameters comprise an at-rest heart rate measured when the user is in a reclined position, an at-rest heart rate measured when the user is in a standing position, a change in at-rest heart rate between when the user is in a reclined position and when the user is in a standing position, an at-rest heart rate variation, orthostatic hypotension, or an intensity of activity, or a combination thereof.

8. The method of claim 7, wherein measuring the at-rest heart rate measured when the user is in a reclined position comprises:
initiating and incrementing an at-rest timer when the user is in the reclined position; and
measuring the user's heart rate when the at-rest timer has met or exceeded a first predetermined at-rest threshold.

9. The method of claim 8, wherein measuring the at-rest heart rate measured when the user is in a standing position comprises:
initiating and incrementing the at-rest timer when the user has transitioned from the reclined position to the standing position; and
measuring the user's heart rate when the at-rest timer has met or exceeded a second predetermined at-rest threshold.

10. The method of claim 1, wherein the one or more subjective user state parameters comprise a value within a numerical range assigned to an average training load over a predetermined period of time, an average training intensity over the predetermined period of time, a quality of sleep, an overall level of life stress, a current level of stress, a quality of food consumed over the predetermined period of time, a quantity of food combination thereof.

11. The method of claim 10, wherein the predetermined period of time is the previous seven days.

12. The method of claim 10, further comprising:
receiving recorded data corresponding to orthostatic hypotension for the user; and
replacing the received user input data corresponding to the level of hydration with the received recorded data corresponding to orthostatic hypotension for the user.

13. A system for determining a readiness of a user, comprising:
a processor configured to:
receive physiological data corresponding to one or more physiological parameters of the user;
receive user input data corresponding to one or more subjective user state parameters of the user;
assign a component score to each of the received physiological data and user input data;
assign a respective weight to at least one of the one or more physiological parameters or one or more subjective user state parameters of the user; and
derive a readiness score for the user based at least in part on the component scores and the corresponding weights; and
a transceiver configured to communicate the derived readiness score to a display device.

14. The system of claim 13, wherein deriving the readiness score further comprises:
calculating a weighted average of the respective component scores for each of the received physiological data and user input data, where the respective component scores are weighted by respective weights.

15. The system of claim 13, wherein the processor is further configured to:
receive recorded data corresponding to the one or more subjective user state parameters of the users; and
replace the received user input data with the received recorded data.

16. The system of claim 13, wherein the received user input data is associated with a value within a numerical range.

17. The system of claim 13, wherein the one or more physiological parameters comprise an at-rest heart rate measured when the user is in a reclined position, an at-rest heart rate measured when the user is in a standing position, a change in at-rest heart rate between when the user is in a reclined position and when the user is in a standing position, an at-rest heart rate variation, orthostatic hypotension, or an intensity of activity, or a combination thereof.

18. The system of claim 13, wherein the one or more subjective user state parameters comprise a value within a numerical range assigned to an average training load over a predetermined period of time, an average training intensity over a predetermined period of time, a quality of sleep, an overall level of life stress, a current level of stress, a quality of food consumed over a predetermined period of time, a quantity of food consumed over a predetermined period of time, a level of pain, or a level of hydration, or a combination thereof.

19. The system of claim 18, wherein the processor is further configured to:
receive recorded data corresponding to orthostatic hypotension for the user; and
replace the received user input data corresponding to the level of hydration with the received recorded data corresponding to orthostatic hypotension for the user.

20. A non-transitory computer-readable medium storing computer-executable code, the code executable by a processor to:
receive physiological data corresponding to one or more physiological parameters of the user;
receive user input data corresponding to one or more subjective user state parameters of the user;
assign a respective component score to each of the received physiological data and user input data;
assign a respective weight to at least one of the one or more physiological parameters or one or more subjective user state parameters of the user;
derive a readiness score for the user based at least in part on the component scores and the corresponding weights; and
communicate the derived readiness score to the user via a display device.
